(19) United States
(12) Patent Application Publication
(10) Pub. No.: US 2022/0375586 A1
(43) Pub. Date: Nov. 24, 2022

(54) SYSTEM, METHOD, AND COMPUTER PROGRAM PRODUCT FOR SMART FLUID INJECTOR PERFORMANCE MONITORING

(71) Applicant: BAYER HEALTHCARE LLC, WHIPPANY, NJ (US)

(72) Inventors: MICHAEL MCDERMOTT, BERLIN (DE); SAMANTHA PARKER, PITTSBURGH, PA (US); ROBERT PRANIEWICZ, NEW KENSINGTON, PA (US); TYLER SKIRBLE, GLENSHAW, PA (US); UTE MCDERMOTT, COLOGNE (DE)

(21) Appl. No.: 17/770,696

(22) PCT Filed: Nov. 19, 2020
(86) PCT No.: PCT/US2020/061161

§ 371 Date: Apr. 21, 2022

Related U.S. Application Data
(60) Provisional application No. 62/938,408, filed on Nov. 21, 2019.

(51) Int. Cl. G16H 40/40; A61M 5/145; G16H 40/63

(52) U.S. Cl. CPC G16H 40/40; A61M 5/1452; G16H 40/63; A61M 2205/332; A61M 2005/14208

(57) ABSTRACT

A method, system, and computer program product are disclosed for performance monitoring of a smart fluid injector system having at least one sensor configured for detecting operation data. The operation data includes one or more operation parameters associated with at least one of: one or more drive components of the fluid injector system and at least one disposable component configured for use with the fluid injector system. The fluid injector system further includes a control device having at least one processor programmed or configured to: receive the operation data, and determine a component status for at least one of: the drive component(s), the disposable component(s), and administration line(s) by comparing the received operation data with stored operation data. The component status includes one or more predictions of an operation failure or a misuse of at least one of: the drive component(s), the disposable component(s), and administration line(s).

### CROSS-REFERENCE TO RELATED APPLICATION

This application claims priority to U.S. Provisional Application No. 62/938,408, filed on Nov. 21, 2019, the disclosure of which is incorporated herein by reference in its entirety.

### BACKGROUND OF THE DISCLOSURE

### Field of the Disclosure

This disclosure relates generally to systems, devices, products, apparatus, and methods that are used for performance monitoring of a smart fluid injector system.

### Description of the Related Art

In many medical diagnostic and therapeutic procedures, a medical practitioner, such as a physician or radiologist, injects a patient with one or more fluids using a powered fluid injector system. In recent years, a number of powered fluid injector systems for pressurized injection of fluids have been developed for use in procedures such as angiography, computed tomography (CT), molecular imaging (such as PET imaging), and magnetic resonance imaging (MM).

Conventional fluid injector systems have no mechanism for predicting operation failures and/or misuses before one or more components or devices of the fluid injector system fail and/or are improperly used. In this way, conventional fluid injector systems may not provide for repairing, servicing, upgrading, and/or replacing before one or more components of the fluid injector systems fails and/or is misused. Availability (e.g., uptime for performing operations, proper functioning and/or performance of operations, non-failure of components, devices, functions, and/or operations, etc.) and proper use (e.g., by a user or operator, etc.) of such fluid injector systems can affect life-saving diagnosis and monitoring of medical treatment for a disease or medical condition of a patient. If one or more components or devices of the fluid injector system fails or is used improperly, imaging can be interrupted and/or a medical procedure and/or treatment for a patient may be delayed and/or improperly performed. Accordingly, there is a need in the art to improve availability and usage of fluid injector systems (e.g., to reduce or prevent downtime, improper functioning, failure, and/or improper use by users or operators of fluid injector systems, etc.).

### SUMMARY OF THE DISCLOSURE

Accordingly, provided are systems, devices, products, apparatus, and/or methods for a smart fluid injector system that improves availability and/or usage of the fluid injector system by predicting operation failures and/or misuses for the fluid injector system before the fluid injector system fails and/or is improperly used and by providing maintenance action associated with the predicted operation failures and/or misuses. In some non-limiting embodiments or aspects, a smart fluid injector system may be configured for gathering data (e.g., performance data sensed by one or more sensors, etc.) that can be used to reduce a risk (e.g., a probability, a likelihood, etc.) of failure and/or misuse of the fluid injector system. In some non-limiting embodiments or aspects, the smart fluid injector system may be further configured for peer-to-peer performance benchmarking of a network of fluid injector systems and determining a risk (e.g., a probability, a likelihood, etc.) of failure and/or misuse of one or more fluid injector systems within the network. In some non-limiting embodiments or aspects, the smart fluid injector system may be further configured for providing a maintenance instruction (e.g., information on correct components and/or devices (e.g., replacement parts, service tools, etc.) and/or operations (e.g., service records, error codes, service procedures, etc.)) to a service technician and/or a user or operator of a fluid injector system for repairing, servicing, upgrading, and/or replacing one or more components of the fluid injector system.

In some non-limiting embodiments or aspects, a fluid injector system may be configured for use in administering at least one fluid to a patient. The fluid injector system may include: at least one sensor configured for detecting operation data during operation of the fluid injector system, wherein the operation data includes one or more operation parameters associated with at least one of: one or more components of the fluid injector system, at least one disposable component configured for use with the fluid injector system, and at least one administration line configured for use with the fluid injector system; and a control device comprising at least one processor programmed or configured to: receive the operation data from the at least one sensor; determine a component status for at least one of: the one or more components of the fluid injector system, the at least one disposable component, and the at least one administration line by comparing the received operation data with stored operation data, wherein the component status includes one or more predictions of an operation failure or a misuse of at least one of: the one or more components of the fluid injector system, the at least one disposable component, and the at least one administration line; and perform at least one action based on the component status deviating from a predetermined threshold.

In some non-limiting embodiments or aspects, the at least one action may include a prompt for a user to initiate at least one maintenance action associated with at least one of: the one or more components of the fluid injector system, the at least one disposable component, and the at least one administration line.

In some non-limiting embodiments or aspects, the at least one maintenance action may include at least one of the following: scheduling a service of the fluid injector system, operating the one or more components of the fluid injector system in a specific manner indicated by the component status, replacing the at least one disposable component, replacing the at least one administration line, or any combination thereof.

In some non-limiting embodiments or aspects, the at least one action may include automatically scheduling at least one maintenance action in response to the component status deviating from the predetermined threshold.

In some non-limiting embodiments or aspects, the at least one action may include automatically stopping operation of the fluid injector system in response to the component status deviating from the predetermined threshold.

In some non-limiting embodiments or aspects, the at least one action may include repeating at least a portion of operation of the fluid injector system during which the component status indicated an operation failure or misuse.

In some non-limiting embodiments or aspects, the at least one processor may be further programmed or configured to store the operation data from repeating at least a portion of operation of the fluid injector system during which the component status indicated an operation failure or misuse.

In some non-limiting embodiments or aspects, the one or more operation parameters may include a viable life rating associated with at least one of the one or more components of the fluid injector system, the at least one disposable component, and the at least one administration line. The one or more processors may be further programmed or configured to: determine the viable life rating based on at least one of the following: number of uses of the at least one disposable component, a force measurement of one or more drive components of the fluid injector system during pressurized delivery of fluid from the at least one disposable component, or any combination thereof.

In some non-limiting embodiments or aspects, the operation failure or misuse of at least one of the one or more components of the fluid injector system, the at least one disposable component, and the at least one administration line may include at least one of the following: failure of an electrical component, failure of a software component, failure of a mechanical component, receiving user input from a user that causes the fluid injector system to operate contrary to one or more predefined operation thresholds, or any combination thereof.

In some non-limiting embodiments or aspects, the at least one sensor may be a temperature sensor, a vibration sensor, a humidity sensor, an acoustic sensor, an optical sensor, an ultrasonic sensor, a load/pressure sensor, a capacitive sensor, a sensor configured for detecting electromagnetic radiation, a user interface configured for accepting a user input, or any combination thereof.

In some non-limiting embodiments or aspects, a computer-implemented method may be provided for monitoring performance of a fluid injector system configured for use in administering at least one fluid to a patient. The method may include receiving, with a control device comprising at least one processor, operation data gathered by at least one sensor during operation of the fluid injector system, wherein the operation data includes one or more operation parameters associated with at least one of: one or more components of the fluid injector system, at least one disposable component configured for use with the fluid injector system, and at least one administration line configured for use with the fluid injector system; determining, with the control device, a component status for at least one of: the one or more components of the fluid injector system, the at least one disposable component, and the at least one administration line by comparing the received operation data with stored operation data, wherein the component status includes one or more predictions of an operation failure or a misuse of at least one of: the one or more components of the fluid injector system, the at least one disposable component, and the at least one administration line; and providing, with the control device, at least one action based on the component status deviating from a predetermined threshold.

In some non-limiting embodiments or aspects, the method may further include prompting a user to initiate at least one maintenance action associated with at least one of: the one or more components of the fluid injector system, the at least one disposable component, and the at least one administration line.

In some non-limiting embodiments or aspects, the at least one maintenance action may include at least one of the following: scheduling a service of the fluid injector system, operating the one or more components of the fluid injector system in a specific manner indicated by the component status, replacing the at least one disposable component, replacing the at least one administration line, or any combination thereof.

In some non-limiting embodiments or aspects, the method may further include automatically scheduling at least one maintenance action in response to the component status deviating from the predetermined threshold.

In some non-limiting embodiments or aspects, the method may further include automatically stopping operation of the fluid injector system in response to the component status deviating from the predetermined threshold.

In some non-limiting embodiments or aspects, the method may further include repeating at least a portion of operation of the fluid injector system during which the component status indicated an operation failure or misuse.

In some non-limiting embodiments or aspects, the method may further include storing the operation data from repeating at least a portion of operation of the fluid injector system during which the component status indicated an operation failure or misuse

In some non-limiting embodiments or aspects, the one or more operation parameters may include a viable life rating associated with the one or more drive components, the at least one disposable component, and the at least one administration line. The one or more processors may be further programmed or configured to: determine the viable life rating based on at least one of the following: number of uses of the at least one disposable component, a force measurement of one or more drive components of the fluid injector system during pressurized delivery of fluid from the at least one disposable component, or any combination thereof.

In some non-limiting embodiments or aspects, the operation failure or misuse of at least one of the one or more components of the fluid injector system, the at least one disposable component, and the at least one administration line may include at least one of the following: failure of an electrical component, failure of a software component, failure of a mechanical component, receiving user input from a user that causes the fluid injector system to operate contrary to one or more predefined operation thresholds, or any combination thereof.

In some non-limiting embodiments or aspects, the at least one sensor may be a temperature sensor, a vibration sensor, a humidity sensor, an acoustic sensor, an optical sensor, an ultrasonic sensor, a load/pressure sensor, a capacitive sensor, a sensor configured for detecting electromagnetic radiation, a user interface configured for accepting a user input, or any combination thereof.

In some non-limiting embodiments or aspects, a computer program product may be provided for monitoring performance of a fluid injector system configured for use in administering at least one fluid to a patient. The computer program product may have at least one non-transitory computer-readable medium having one or more instructions that, when executed by at least one processor, may cause the at least one processor to: receive operation data gathered by at least one sensor during operation of the fluid injector system, wherein the operation data includes one or more operation parameters associated with at least one of: one or more drive components of the fluid injector system, at least one disposable component configured for use with the fluid injector system, and at least one administration line configured for use with the fluid injector system; determine a component status for at least one of: the one or more drive components and the at least one disposable component by comparing the received operation data with stored operation data, wherein the component status includes one or more predictions of an operation failure or a misuse of at least one of: the one or more drive components, the at least one disposable component, and the at least one administration line; and perform at least one action based on the component status deviating from a predetermined threshold.

In some non-limiting embodiments or aspects, the at least one action may include a prompt for a user to initiate at least one maintenance action associated with at least one of: the one or more drive components and the at least one disposable component.

In some non-limiting embodiments or aspects, the at least one maintenance action may include at least one of the following: scheduling a service of the fluid injector system, operating the one or more drive components of the fluid injector system in a specific manner indicated by the component status, replacing the at least one disposable component, replacing the at least one administration line, or any combination thereof.

In some non-limiting embodiments or aspects, the at least one action may include automatically scheduling at least one maintenance action in response to the component status deviating from the predetermined threshold.

In some non-limiting embodiments or aspects, the at least one action may include automatically stopping operation of the fluid injector system in response to the component status deviating from the predetermined threshold.

In some non-limiting embodiments or aspects, the at least one action may include repeating at least a portion of operation of the fluid injector system during which the component status indicated an operation failure or misuse.

In some non-limiting embodiments or aspects, the one or more instructions, when executed by the at least one processor, further may cause the at least one processor to store the operation data from repeating at least a portion of operation of the fluid injector system during which the component status indicated an operation failure or misuse.

In some non-limiting embodiments or aspects, the one or more operation parameters may include a viable life rating associated with at least one of the one or more components of the fluid injector system, the at least one disposable component, and the at least one administration line. The one or more processors may be further programmed or configured to: determine the viable life rating based on at least one of the following: number of uses of the at least one disposable component, a force measurement of one or more drive components of the fluid injector system during pressurized delivery of fluid from the at least one disposable component, or any combination thereof.

In some non-limiting embodiments or aspects, the operation failure or misuse of at least one of the one or more components of the fluid injector system, the at least one disposable component, and the at least one administration line may include at least one of the following: failure of an electrical component, failure of a software component, failure of a mechanical component, receiving user input from a user that causes the fluid injector system to operate contrary to one or more predefined operation thresholds, or any combination thereof.

In some non-limiting embodiments or aspects, the at least one sensor may be a temperature sensor, a vibration sensor, a humidity sensor, an acoustic sensor, an optical sensor, an ultrasonic sensor, a load/pressure sensor, a capacitive sensor, a sensor configured for detecting electromagnetic radiation, a user interface configured for accepting a user input, or any combination thereof.

Further non-limiting embodiments or aspects are set forth in the following numbered clauses:

Clause 1. A fluid injector system configured for use in administering at least one fluid to a patient, the fluid injector system comprising: at least one sensor configured for detecting operation data during operation of the fluid injector system, wherein the operation data includes one or more operation parameters associated with at least one of: one or more components of the fluid injector system, at least one disposable component configured for use with the fluid injector system, and at least one administration line configured for use with the fluid injector system; and a control device comprising at least one processor programmed or configured to: receive the operation data from the at least one sensor; determine a component status for at least one of: the one or more components of the fluid injector system, the at least one disposable component, and the at least one administration line by comparing the received operation data with stored operation data, wherein the component status includes one or more predictions of an operation failure or a misuse of at least one of: the one or more components of the fluid injector system, the at least one disposable component, and the at least one administration line; and perform at least one action based on the component status deviating from a predetermined threshold.

Clause 2. The fluid injector system of clause 1, wherein the at least one action includes a prompt for a user to initiate at least one maintenance action associated with at least one of: the one or more components of the fluid injector system, the at least one disposable component, and the at least one administration line.

Clause 3. The fluid injector system of clause 2, wherein the at least one maintenance action includes at least one of the following: scheduling a service of the fluid injector system, operating the one or more components of the fluid injector system in a specific manner indicated by the component status, replacing the at least one disposable component, replacing the at least one administration line, or any combination thereof.

Clause 4. The fluid injector system of any of clauses 1-3, wherein the at least one action includes at least one of: automatically scheduling at least one maintenance action in response to the component status deviating from the predetermined threshold; automatically stopping operation of the fluid injector system in response to the component status deviating from the predetermined threshold; and repeating at least a portion of operation of the fluid injector system during which the component status indicated an operation failure or misuse with the least one processor being further programmed or configured to store the operation data detected by the at least one sensor during the repetition of the at least a portion of operation of the fluid injector system.

Clause 5. The fluid injector system of any of clauses 1-4, wherein the one or more operation parameters include a viable life rating associated with at least one of the one or more components of the fluid injector system, the at least one disposable component, and the at least one administration line, and wherein the one or more processors are further programmed or configured to: determine the viable life rating based on at least one of the following: number of uses of the at least one disposable component, a force measurement of one or more drive components of the fluid injector system during pressurized delivery of fluid from the at least one disposable component, or any combination thereof.

Clause 6. The fluid injector system of any of clauses 1-5, wherein the operation failure or misuse of at least one of the one or more components of the fluid injector system, the at least one disposable component, and the at least one administration line includes at least one of the following: failure of an electrical component, failure of a software component, failure of a mechanical component, receiving user input from a user that causes the fluid injector system to operate contrary to one or more predefined operation thresholds, or any combination thereof.

Clause 7. The fluid injector system of any of clauses 1-6, wherein the at least one sensor is a temperature sensor, a vibration sensor, a humidity sensor, an acoustic sensor, an optical sensor, an ultrasonic sensor, a load/pressure sensor, a capacitive sensor, a sensor configured for detecting electromagnetic radiation, a user interface configured for accepting a user input, or any combination thereof.

Clause 8. A computer-implemented method for monitoring performance of a fluid injector system configured for use in administering at least one fluid to a patient, the method comprising: receiving, with a control device comprising at least one processor, operation data gathered by at least one sensor during operation of the fluid injector system, wherein the operation data includes one or more operation parameters associated with at least one of: one or more components of the fluid injector system, at least one disposable component configured for use with the fluid injector system, and at least one administration line configured for use with the fluid injector system; determining, with the control device, a component status for at least one of: the one or more components of the fluid injector system, the at least one disposable component, and the at least one administration line by comparing the received operation data with stored operation data, wherein the component status includes one or more predictions of an operation failure or a misuse of at least one of: the one or more components of the fluid injector system, the at least one disposable component, and the at least one administration line; and providing, with the control device, at least one action based on the component status deviating from a predetermined threshold.

Clause 9. The computer-implemented method of clause 8, further comprising prompting a user to initiate at least one maintenance action associated with at least one of: the one or more components of the fluid injector system, the at least one disposable component, and the at least one administration line.

Clause 10. The computer-implemented method of clause 9, wherein the at least one maintenance action includes at least one of the following: scheduling a service of the fluid injector system, operating the one or more components of the fluid injector system in a specific manner indicated by the component status, replacing the at least one disposable component, replacing the at least one administration line, or any combination thereof.

Clause 11. The computer-implemented method of any of clauses 8-10, further comprising at least one of: automatically scheduling at least one maintenance action in response to the component status deviating from the predetermined threshold; automatically stopping operation of the fluid injector system in response to the component status deviating from the predetermined threshold; and repeating at least a portion of operation of the fluid injector system during which the component status indicated an operation failure or misuse and storing the operation data resulting therefrom.

Clause 12. The computer-implemented method of any of clauses 8-11, wherein the one or more operation parameters include a viable life rating associated with the one or more drive components, the at least one disposable component, and the at least one administration line, and wherein the one or more processors are further programmed or configured to: determine the viable life rating based on at least one of the following: number of uses of the at least one disposable component, a force measurement of one or more drive components of the fluid injector system during pressurized delivery of fluid from the at least one disposable component, or any combination thereof.

Clause 13. The computer-implemented method of any of clauses 8-12, wherein the operation failure or misuse of at least one of the one or more components of the fluid injector system, the at least one disposable component, and the at least one administration line includes at least one of the following: failure of an electrical component, failure of a software component, failure of a mechanical component, receiving user input from a user that causes the fluid injector system to operate contrary to one or more predefined operation thresholds, or any combination thereof.

Clause 14. The computer-implemented method of any of clauses 8-13, wherein the at least one sensor is a temperature sensor, a vibration sensor, a humidity sensor, an acoustic sensor, an optical sensor, an ultrasonic sensor, a load/pressure sensor, a capacitive sensor, a sensor configured for detecting electromagnetic radiation, a user interface configured for accepting a user input, or any combination thereof.

Clause 15. A computer program product for monitoring performance of a fluid injector system configured for use in administering at least one fluid to a patient, the computer program product comprising at least one non-transitory computer-readable medium comprising one or more instructions that, when executed by at least one processor, cause the at least one processor to: receive operation data gathered by at least one sensor during operation of the fluid injector system, wherein the operation data includes one or more operation parameters associated with at least one of: one or more drive components of the fluid injector system, at least one disposable component configured for use with the fluid injector system, and at least one administration line configured for use with the fluid injector system; determine a component status for at least one of: the one or more drive components and the at least one disposable component by comparing the received operation data with stored operation data, wherein the component status includes one or more predictions of an operation failure or a misuse of at least one of: the one or more drive components, the at least one disposable component, and the at least one administration line; and perform at least one action based on the component status deviating from a predetermined threshold.

Clause 16. The computer program product of clause 15, wherein the at least one action includes a prompt for a user to initiate at least one maintenance action associated with at least one of: the one or more drive components and the at least one disposable component.

Clause 17. The computer program product of clause 16, wherein the at least one maintenance action includes at least one of the following: scheduling a service of the fluid injector system, operating the one or more drive components of the fluid injector system in a specific manner indicated by the component status, replacing the at least one disposable component, replacing the at least one administration line, or any combination thereof.

Clause 18. The computer program product of any of clauses 15-17, wherein the at least one action includes at least one of: automatically scheduling at least one maintenance action in response to the component status deviating from the predetermined threshold; automatically stopping operation of the fluid injector system in response to the component status deviating from the predetermined threshold; and repeating at least a portion of operation of the fluid injector system during which the component status indicated an operation failure or misuse with the one or more instructions, when executed by the at least one processor, further causing the at least one processor to store the operation data resulting therefrom.

Clause 19. The computer program product of any of clauses 15-18, wherein the one or more operation parameters include a viable life rating associated with at least one of the one or more components of the fluid injector system, the at least one disposable component, and the at least one administration line, and wherein the one or more processors are further programmed or configured to: determine the viable life rating based on at least one of the following: number of uses of the at least one disposable component, a force measurement of one or more drive components of the fluid injector system during pressurized delivery of fluid from the at least one disposable component, or any combination thereof.

Clause 20. The computer program product of any of clauses 15-19, wherein the operation failure or misuse of at least one of the one or more components of the fluid injector system, the at least one disposable component, and the at least one administration line includes at least one of the following: failure of an electrical component, failure of a software component, failure of a mechanical component, receiving user input from a user that causes the fluid injector system to operate contrary to one or more predefined operation thresholds, or any combination thereof.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure. As used in the specification and the claims, the singular form of “a,” “an,” and “the” include plural referents unless the context clearly dictates otherwise.

### BRIEF DESCRIPTION OF THE DRAWINGS

Additional advantages and details of the disclosure are explained in greater detail below with reference to the exemplary embodiments or aspects that are illustrated in the accompanying schematic figures, in which:

FIG. 1A is a perspective view of a fluid injector system according to some embodiment or aspect of the present disclosure;

FIG. 1B is a perspective view of a single-use disposable set configured for connecting to a fluid injector system in accordance with some examples of the present disclosure;

FIG. 2 is a perspective view of a multi-use disposable set for use with the fluid injector system of FIG. 1;

FIG. 3 is a perspective view of a fluid injector system according to another embodiment or aspect of the present disclosure;

FIG. 4 is a schematic view of an electronic control system of a fluid injector system in accordance with additional embodiments or aspects of the present disclosure; and

FIG. 5 is a flowchart of one non-limiting embodiment or aspect of a process for monitoring performance of a fluid injector system.

### DETAILED DESCRIPTION OF THE DISCLOSURE

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures.

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a fluid reservoir, such as a syringe, a rolling diaphragm, or multiple syringe disposable set, the term “distal” refers to a portion of the fluid reservoir nearest to a patient. When used in relation to a fluid reservoir, such as a syringe, a rolling diaphragm, or multiple syringe disposable set, the term “proximal” refers to a portion of the fluid reservoir nearest to the injector system.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “server” may refer to one or more computing devices, such as processors, storage devices, and/or similar computer components that communicate with client devices and/or other computing devices over a network, such as the Internet or private networks, and, in some examples, facilitate communication among other servers and/or client devices. It will be appreciated that various other arrangements are possible. As used herein, the term “system” may refer to one or more computing devices or combinations of computing devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

Non-limiting embodiments or aspects of the present disclosure are directed to systems, devices, products, apparatus, and/or methods for monitoring performance of a fluid injector system that improves availability and/or usage of the fluid injector system by predicting operation failures and/or misuses before the one or more components of the fluid injector system fail and/or are improperly used. In this way, various embodiments or aspects of the present disclosure provide for: (I) repairing, servicing, upgrading, and/or replacing (e.g., scheduling and/or performing a repair, service, upgrade, and/or replacement, etc.) of a fluid injector system (e.g., one or more components or devices of the fluid injector system) before the fluid injector system (e.g., before one or more components or devices of the fluid injector system) fails and/or is misused, which can (a) decrease or prevent downtime of the fluid injector system, (b) increase a lifetime of the fluid injector system (e.g., decrease a time to a failure requiring replacement of the fluid injector system, etc.), (c) increase a number and/or a likelihood of successful procedures and/or treatments for patients, (d) increase efficiency in scheduling and/or performance of repairs, services, upgrades and/or replacements (e.g., automatically schedule and/or perform a repair, service, upgrade, and/or replacement with an fluid injector system, automatically provide a prompt for a user to schedule and/or perform a repair, service, upgrade, and/or replacement of a fluid injector system, etc.), or the like; (II) providing maintenance action (e.g., maintenance actions, training information, etc.) for a user or operator of the fluid injector system that the user or operator can implement to reduce a risk (e.g., a probability, a likelihood, etc.) of failure and/or misuse of the fluid injector system, which can (a) decrease or prevent continued misuse of the fluid injector system, (b) increase image quality and/or patient care (e.g., decrease an occurrence of repeat injections and/or scans, etc.), (c) decrease contrast waste, and/or the like; (III) providing maintenance action (e.g., information on components and/or devices (e.g., replacement parts, service tools, etc.) and/or operations (e.g., service records, error codes, service procedures, etc.)) to a service technician and/or a user or operator of an fluid injector system for repairing, servicing, upgrading, and/or replacing the fluid injector system, which can (a) ensure that a service technician has the correct parts, tools, and/or information for a particular repair, service, upgrade, and/or replacement, (b) improve efficiency in scheduling of multiple repairs, services, upgrades and/or replacements, and/or the like; (IV) automatically ensuring regulatory compliance of a fluid injector system before the fluid injector system becomes incompliant with one or more regulations, which can (a) ensure calibration settings of the fluid injector system, (b) reduce patient infections, (c) improve cleanliness of the fluid injector system, and/or the like; (V) service benchmarking of inventory systems, which can (a) provide information for warranty prediction, service inventory planning, service resource planning, etc. that a user or customer can use to improve a knowledge base for preventative maintenance, (b) define use cases for future products, (c) improve training focus areas, and/or the like; and/or the like.

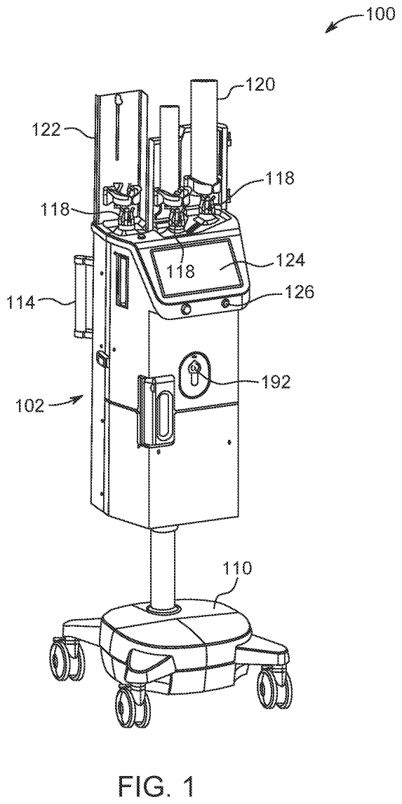

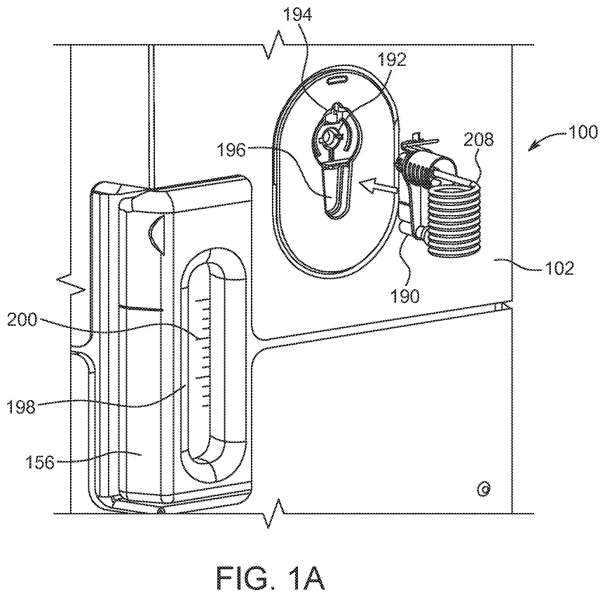

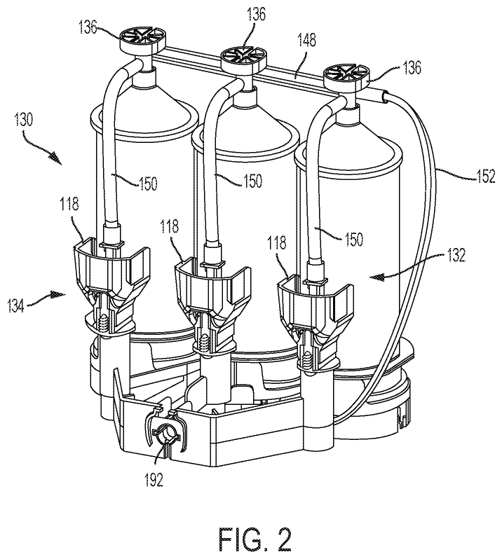

Referring to the drawings in which like reference characters refer to like parts throughout the several views thereof, one aspect or example of the present disclosure is generally directed to a multi-fluid medical injector/injector system 100 (hereinafter “fluid injector system 100”) which in certain embodiments may include a multi-use disposable set (MUDS) 130 configured for delivering fluid to a patient using a single-use disposable set (SUDS) 190 (shown in FIG. 1B) and in other embodiments may include two or more disposable fluid reservoirs or syringes, which may be disposed of after one injection procedure or a specific number of injection procedures. The fluid injector system 100 may include multiple components as individually described herein. Generally, the fluid injector system 100 depicted in FIGS. 1A-1B has a powered injector or other administration device and a fluid delivery set intended to be associated with the injector to deliver one or more fluids from one or more multi-dose containers under pressure into a patient, as described herein. The various devices, components, and features of the fluid injector system 100 and the fluid delivery set associated therewith are likewise described in detail herein. While the various examples of the methods and processes are shown with reference to an injector system having a multi-use disposable set (“MUDS”) and a single-use disposable set (“SUDS”) configuration in FIGS. 1A, 1B, and 2, the disclosure is not limited to such an injector system and may be utilized in other syringe based injector systems, such as but not limited to those described in U.S. Pat. Nos. 7,553,294; 7,563,249; 8,945,051; 9,173,995; 10,124,110; 10, 507,319; 10,583,256; and U.S. Application Publication No. 2018/0161496, the disclosures of each of which are incorporated herein in their entirety by this reference.

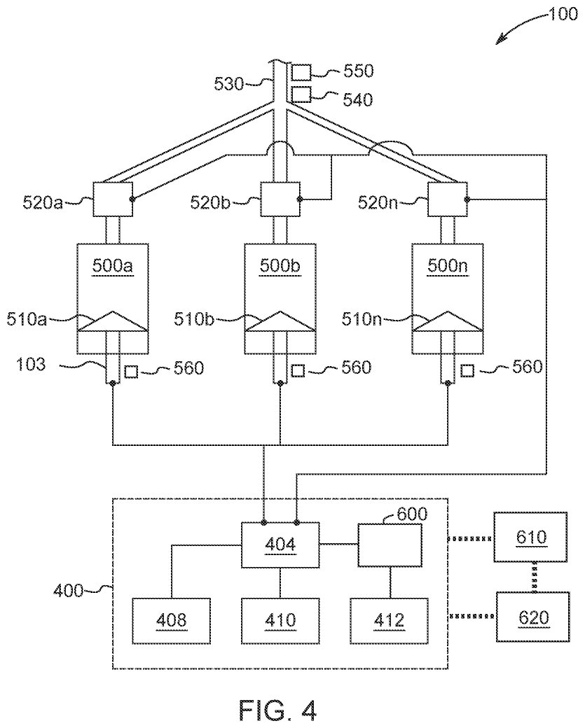

With reference to FIG. 1A, the fluid injector system 100 according to one embodiment or aspect includes an injector housing 102 that encloses the various mechanical drive components, electrical and power components necessary to drive the mechanical drive components, and control components, such as electronic memory and electronic control devices, used to control operation of reciprocally movable pistons (see, for example, drive components 510a-510n in FIG. 4) associated with the fluid injector system 100 described herein. Such pistons may be reciprocally operable via electro-mechanical drive components such as a ball screw shaft driven by a motor, a voice coil actuator, a rack-and-pinion gear drive, a linear motor, and the like.

The fluid injector system 100 may include at least one bulk fluid connector 118 for connection with at least one bulk fluid source 120. In some examples, a plurality of bulk fluid connectors 118 may be provided. For example, as shown in the fluid injector embodiment illustrated in FIG. 1A, three bulk fluid connectors 118 may be provided in a side-by-side or other arrangement. In some examples, the at least one bulk fluid connector 118 may include a spike configured for removably connecting to the at least one bulk fluid source 120, such as a vial, a bottle, or a bag. The at least one bulk fluid connector 118 may be formed on the multi-use disposable set (“MUDS”), as described herein. The at least one bulk fluid source 120 may be configured for receiving a medical fluid, such as saline, Ringer's lactate, an imaging contrast medium solution, or other medical fluid, for delivery to the patient by the fluid injector system 100.

With reference to FIG. 2, a MUDS 130 is configured for being removably connected to the fluid injector system 100 for delivering one or more fluids from the one or more bulk fluid sources 120 to the patient. Examples and features of embodiments of the MUDS are further described in PCT International Publication No. WO 2016/112163, filed on Jan. 7, 2016, the disclosure of which is incorporated herein by reference in its entirety. The MUDS 130 may include one or more fluid reservoirs, such as one or more syringes 132. As used herein, the term “fluid reservoir” means any container capable of taking in and delivering a fluid, for example during a fluid injection procedure including, for example, a syringe, a rolling diaphragm, a pump, a compressible bag, and the like. Fluid reservoirs may include the interior volume of at least a portion of a fluid pathway, such as one or more tubing lengths, that are in fluid communication with the interior of the fluid reservoir, including fluid pathway portions that remain in fluid communication with the fluid reservoir after the system is closed or fluidly isolated from the remainder of the fluid pathway. In some examples, the number of fluid reservoirs may correspond to the number of bulk fluid sources 120 (shown in FIG. 1A). For example, with reference to FIG. 2, the MUDS 130 has three syringes 132 in a side-by-side arrangement such that each syringe 132 is fluidly connectable to one or more of the corresponding three bulk fluid sources 120. In some examples, one or more bulk fluid sources 120 may be connected to one or more syringes 132 of the MUDS 130. Each syringe 132 may be fluidly connectable to one of the bulk fluid sources 120 by a corresponding bulk fluid connector 118 and an associated MUDS fluid path 134. The MUDS fluid path 134 may have a spike element that connects to the bulk fluid connector 118 and the fluid line 150. In some examples, the bulk fluid connector 118 may be provided directly on the MUDS 130. In some non-limiting embodiments or aspects, the MUDS 130 may define the at least one disposable component of the fluid injector system 100, as discussed herein.

With continued reference to FIGS. 1A and 2, the MUDS 130 may include one or more valves 136, such as stopcock valves, for controlling which medical fluid or combinations of medical fluids are withdrawn from the multi-dose bulk fluid source 120 (see FIG. 1A) into the fluid reservoirs 132 and/or are delivered to a patient from each fluid reservoir 132. In some examples, the one or more valves 136 may be provided on a distal end of the plurality of syringes 132 or on a manifold 148. The manifold 148 may be in selectable fluid communication via valves 136 with the interior volume of the syringes 132. The interior volume of the syringes 132 may be in selectable fluid communication via valves 136 with a first end of the MUDS fluid path 134 that connects each syringe 132 to the corresponding bulk fluid source 120. The opposing second end of the MUDS fluid path 134 may be connected to the respective bulk fluid connector 118 that is configured for fluidly connecting with the bulk fluid source 120. Depending on the position of the one or more valves 136, fluid may be drawn into the interior volume of the one or more syringes 132 or it may be delivered from the interior volume of the one or more syringes 132. In a first position, such as during the filling of the syringes 132, the one or more valves 136 are oriented such that fluid flows from the bulk fluid source 120 into the desired syringe 132 through a fluid inlet line 150, such as a MUDS fluid path. During the filling procedure, the one or more valves 136 are positioned such that fluid flow through one or more fluid outlet lines 152 or manifold 148 is blocked or closed. In a second position, such as during a fluid delivery procedure, fluid from one or more syringes 132 is delivered to the manifold 148 through the one or more fluid outlet lines 152 or syringe valve outlet ports. During the fluid delivery procedure, the one or more valves 136 are positioned such that fluid flow through one or more fluid inlet lines 150 is blocked or closed. In a third position, the one or more valves 136 are oriented such that fluid flow through the one or more fluid inlet lines 150 and the one or more fluid outlet lines 152 or manifold 148 is blocked or closed. Thus, in the third position, each of the one or more valves 136 isolates the corresponding syringe 132 and prevents fluid flow into and out of the interior volume of the corresponding syringe 132. As such, each of the one or more syringes 132 and the corresponding valve 136 defines a closed system.

The one or more valves 136, fluid inlet lines 150, and/or fluid outlet lines 152 may be integrated into or in fluid communication via the manifold 148. The one or more valves 136 may be selectively positioned to the first or second position by manual or automatic handling. For example, the operator may position the one or more valves 136 into the desired position for filling, fluid delivery, or the closed position. In other examples, at least a portion of the fluid injector system 100 is operable for automatically positioning the one or more valves 136 into a desired position for filling, fluid delivery, or the closed position based on input by the operator or by a protocol executed by the electronic control unit.

With continued reference to FIGS. 1A, 1B, and 2, according to the described embodiment, the fluid injector system 100 may have a connection port 192 that is configured to form a releasable fluid connection with at least a portion of the SUDS 190. In some examples, the connection port 192 may be formed on the MUDS 130. As described herein, the SUDS 190 may be connected to the connection port 192, formed on at least a portion of the MUDS 130 and/or the housing 102. Desirably, the connection between the SUDS 190 and the connection port 192 is a releasable connection to allow the SUDS 190 to be selectively connected to and disconnected from the connection port 192. In some examples, the SUDS 190 may be disconnected from the connection port 192 and disposed after each fluid delivery procedure, and a new SUDS 190 may be connected to the connection port 192 for a subsequent fluid delivery procedure. The SUDS 190 may be used to deliver one or more medical fluids to a patient by SUDS fluid line 208 having a distal end that may be selectively disconnected from the body of the SUDS 190 and connected to a patient catheter. Other examples and features of the SUDS 190 are described in U.S. Pat. No. 10,549,084, the disclosure of which is incorporated herein by reference in its entirety.

Referring again to FIG. 1A, the fluid injector system 100 may include one or more user interfaces 124, such as a graphical user interface (GUI) display window. The user interface 124 may display information pertinent to a fluid injection procedure involving fluid injector system 100, such as injection status or progress, current flow rate, fluid pressure, and volume remaining in the at least one bulk fluid source 120 connected to the fluid injector system 100 and may be a touch screen GUI that allows an operator to input commands and/or data for operation of fluid injector system 100. In some non-limiting embodiments or aspects, the user interface 124 may be configured for displaying information performance and/or maintenance action regarding at least one component of the fluid injector system 100. Additionally, the fluid injector system 100 and/or user interface 124 may include at least one control button 126 for tactile operation by an attendant operator of the fluid injector system 100. The at least one control button 126 may be a graphical part of the user interface 124, such as a touch screen.

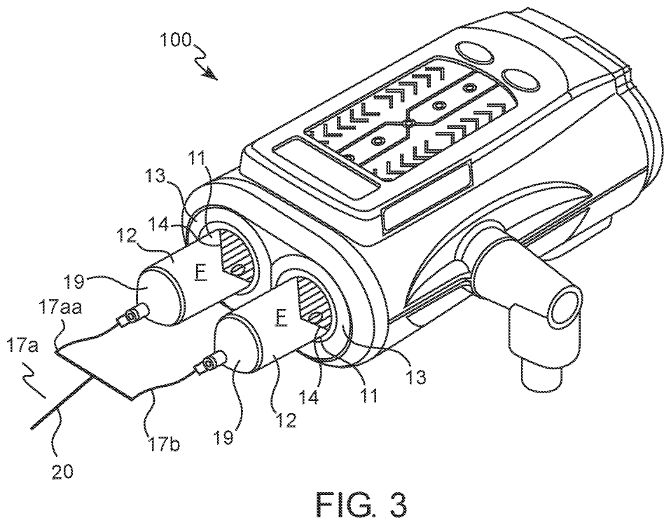

While FIGS. 1A, 1B, and 2 illustrate one example of a fluid injector system 100 and associated components and structure, it is to be understood that the present disclosure is not limited to any particular type or variety of the fluid injector system 100. Referring now to FIG. 3, another non-limiting example of a fluid injector system 100 in accordance with the present disclosure includes at least one fluid reservoir, such as syringe 12, at least one piston (see, for example, drive components 510a-510n in FIG. 4) connectable to at least one plunger 14, and a fluid control module (not pictured). The at least one syringe 12 is generally adapted to interface with at least one component of the system, such as a syringe port 13. The fluid injector system 100 is generally configured to deliver at least one fluid F to a patient during an injection procedure. The fluid injector system 100 is configured to releasably receive the at least one syringe 12, which is to be filled with at least one fluid F, such as a contrast media, saline solution, Ringer's lactate, or any desired medical fluid. The system may be a multi-syringe injector, wherein several syringes may be oriented side-by-side or in another spatial relationship and are separately actuated by respective pistons associated with the injector. The at least one syringe 12 may be oriented in any manner such as upright, downright, or positioned at any degree angle. In another embodiment, a fluid injector 100 may interface with one or more rolling diaphragm syringes (not shown). Non-limiting examples of rolling diaphragm syringe-based injectors are described in U.S. Pat. No. 10,583,256, U.S. Patent Application Publication No. 2018/0161496, and International Application Publication No. WO 2018/075386, the disclosures of which are incorporated herein.

With continued reference to FIG. 3, the injector system 100 may be used during a medical procedure to inject the at least one medical fluid F into the vasculature system of a patient by driving a plunger 14 of at least one syringe 12 with a drive member, such as the at least one piston (see, for example, drive components 510a-510n in FIG. 4). The at least one piston may be reciprocally operable upon at least a portion of the at least one syringe, such as the plunger 14. Upon engagement, the at least one piston may move the plunger 14 toward the distal end 19 of the at least one syringe, as well as retracting the plunger 14 toward the proximal end 11 of the at least one syringe 12.

A tubing set 17 (e.g., first and second fluid conduits 17a and 17b, and common administration line 20) may be in fluid communication with an outlet port of each syringe 12 to place each syringe in fluid communication with a catheter for delivering the fluid F from each syringes 12 to the catheter (not shown) inserted into a patient at a vascular access site. The first and second fluid conduits 17a and 17b may be connected to the common administration line 20 by any suitable mechanism known in the art (e.g., a Y-connector or a T-connector). The fluid injector system 100 shown in FIG. 3 is an open system due to the lack of valves capable of isolating the syringes 12 from one another and from at least a portion of the tubing set 17. However, it is to be understood that valves, similar or identical to the valves 136 described with reference to the fluid injector system 100 of FIGS. 1 and 2, may be added distally of the syringes 12 to convert the fluid injector system 100 of FIG. 3 to a closed system.

Referring now to FIG. 4, fluid injector systems 100 in accordance with the present disclosure may be associated with and controlled by an electronic control device 400 configured to execute one or more injector protocols including, for example, the filling, priming, and delivery operations. In some examples or aspects, the electronic control device 400 further may be configured to receive operation data gathered by one or more sensors, as discussed herein. In some examples, the electronic control device 400 may control the operation of various valves, stopcocks, piston members, and other elements to affect a desired gas/air removal or purge, filling, and/or delivery procedure. In some examples, the electronic control device 400 may be configured to gather and process operation data, such as one or more operation parameters associated with at least one disposable component configured for use with the fluid injector system 100. As discussed herein, operation data may include, for example, pressure data from a load sensor, voltage data from an optical sensor, digital output from an ultrasonic sensor, analog to digital converted counts from any sensor of the fluid injector system 100, and/or collected data on buttons pressed or information entered on a user interface. Operation data further may include dormant state data collected while the fluid injector system 100 is not in use. Operation data further may include calibration data collected during maintenance and calibration procedures performed on the fluid injector system 100. Operation data may be in the form of a voltage, current, ADC value, or any other form that is indicative of an operating status of one or more components of the fluid injector system 100. Operation data further may include alerts initiated by the fluid injector system 100, and/or stored data of user interactions with the fluid injector system 100, such as interactions with the graphical user interface, the sequence of buttons that are pressed or sequences that are initiated during operation of the fluid injector system 100.

The electronic control device 400 may include at least one processor 404, memory 408, an input component 410, and an output component 412. The electronic control device 400 further may include a bus that permits communication among the components of electronic control device 400. The at least one processor 404 may be implemented in hardware, firmware, or a combination of hardware and software. For example, processor 404 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 408 may include a hard disk (e.g., a magnetic disk, an optical disk, a magneto-optic disk, a solid state disk, etc.) and/or another type of computer-readable medium. The input component 410 may include a component that permits the electronic control device 400 to receive information, such as via user input (e.g., the user interface 124) and/or information from one or more sensors 414. Additionally, or alternatively, the input component 410 may include one or more sensors for sensing information (e.g., a global positioning system (GPS) component, an accelerometer, a gyroscope, an actuator, etc.). The output component 412 may include a component that provides output information from the electronic control device 400 (e.g., the user interface 124).

The electronic control device 400 may be programmed or configured to perform one or more processes and/or methods based on the at least one processor 404 executing software instructions stored by a computer-readable medium, such as memory 408. When executed, software instructions stored in memory 408 may cause the at least one processor 404 to perform one or more processes and/or methods described herein. In some examples or aspects, the at least one processor 404 may be configured to collect data continuously and/or periodically from one or more components of the fluid injector system 100 capable of providing an electrical signal, analog data, and/or digital data. The collected data may be stored locally, such as in the memory 408, for determination of actions to be taken. Alternatively, or in addition, the collected data may be sent to a separate processing unit for analysis and decision making to occur. Data may be stored in a database, a table, a library, or any other form of storage. The analysis includes making determinations about one or more components of the fluid injector system 100, including those that are unable to send or receive data.

With continued reference to FIG. 4, the electronic control device 400, more particularly the at least one processor 404, may be in operative communication with one or more components of the fluid injector system 100 via one or more sensors to monitor the performance of the fluid injector system 100. The electronic control device 400 may be in operative communication with one or more drive components 510a, 510b, 510n, such as via one or more sensors, respectively associated with one or more fluid reservoirs 500a, 500b, 500n of the fluid injector system 100. More particularly, each of the one or more drive components 510a, 510b, 510n may be associated with one of the fluid reservoirs 500a, 500b, 500n such that fluid contained in each of the fluid reservoirs 500a, 500b, 500n may be selectively delivered via actuation of the associated drive component 510a, 510b, 510n. The fluid reservoirs 500a, 500b, 500n may be, or may correspond to, the syringes 132 of the fluid injector system 100 of FIGS. 1A-2 and/or the syringes 12 of the fluid injector system 100 of FIG. 3 or other syringe-type structures, such as rolling diaphragm syringes, as described herein. The one or more drive components 510a, 510b, 510n may be, or may correspond to, the pistons of the fluid injector systems 100 of FIGS. 1A-3. In other embodiments or aspects, the one or more drive components 510a, 510b, 510n may be pumps, such as peristaltic pumps, or other fluid delivery devices configured for delivering fluid from one or more of the fluid reservoirs 500a, 500b, 500n to a patient. The one or more fluid reservoirs 500a, 500b, 500n may be in fluid communication with an administration line 530 for delivering fluid to a catheter or other component connected to a patient. The administration line 530 may be, or may correspond to, the SUDS 190 of the fluid injector system 100 of FIGS. 1A, 1B, and 2 and/or the tubing set 17 of the fluid injector system 100 of FIG. 3.

In aspects and examples of a closed fluid injector system 100 (e.g., the fluid injector system 100 of FIGS. 1A and 2), the electronic control device 400 further may be in operative communication with one or more valves 520a, 520b, 520n, such as via one or more sensors, in order to rotate or otherwise actuate the valves 520a, 520b, 520n to direct flow into or out of and/or isolate flow from one or more of the fluid reservoirs 500a, 500b, 500n to the administration line 530. The valves 520a, 520b, 520n may be, or may correspond to, the valves 136 described herein in connection with FIG. 2.

In some non-limiting embodiments or aspects, the fluid injector system 100 may have one or more flow rate sensors 540 for directly measuring a flow rate and/or a volume of a fluid flow. For example, the one or more flow rate sensors 540 (e.g., an ultrasonic mass flow rate sensor) may be provided near an outlet of the administration line 530 and may be configured to measure a flow rate and/or a volume of a fluid flow. In such an example, the one or more flow rate sensors 540 can be configured to directly measure a flow rate of fluid flowing through the administration line 530 and/or a volume of the fluid flowing through the administration line 530 (e.g., a total volume delivered for an injection, etc.). The one or more flow rate sensors 540 can measure the flow rate and/or volume of a fluid flow in the administration line 530, and/or the flow rate and/or volume of a fluid flow out of the one or more of the fluid reservoirs 500a, 500b, 500n, which is controlled and/or provided by the one or more drive components of the fluid injector system 100 (e.g., a pump powered by a motor, etc.), such as a positive displacement pump, a non-positive displacement pump, a semi-positive displacement pump, a reciprocating pump, a piston pump, a vane pump, a flexible member pump, a lobe pump, a gear pump, a circumferential piston pump, a screw pump, a centrifugal pump, a turbine pump, an impeller pump, and/or the like. In some non-limiting embodiments or aspects, the one or more flow rate sensors 540 provide a real-time feedback signal via a feedback control loop between the one or more flow rate sensors 540 and the electronic control device 400 of the fluid injector system 100. The fluid injector system 100 is configured to compare a flow rate measurement of a current or more recent injection to flow rate measurements of previous injections or a calibration measurement based on a same injection protocol to determine the extent to which the flow rate has changed from the current or most recent injection to that associated with previous injection(s) or the calibration measurement.

In some non-limiting embodiments or aspects, the fluid injector system 100 may have one or more air sensors 550 configured to detect air or gas in a fluid flow. For example, the one or more air sensors 550 can be configured to directly measure an amount of air or gas in the fluid flowing through the administration line 530. In some non-limiting embodiments or aspects, the one or more air sensors 550 provide a real-time feedback signal via a feedback control loop between the one or more air sensors 550 and the electronic control device 400 of the fluid injector system 100.

In some non-limiting embodiments or aspects, the fluid injector system 100 may have one or more force sensors 560, (e.g., a motor current sensor, a strain gauge, etc.) configured to measure a force exerted by the one or more drive components 510a, 510b, 510n in order to move the plungers within the one or more of the fluid reservoirs 500a, 500b, 500n. In some non-limiting embodiments or aspects, the one or more force sensors 560 provide a real-time feedback signal via a feedback control loop between the one or more force sensors 560 and the electronic control device 400 of the fluid injector system 100. The fluid injector system 100 is configured to compare a force measurement of a current or more recent injection to force measurements of previous injections or a calibration measurement to determine the extent to which the force required to move the one or more drive components 510a, 510b, 510n has changed from the current or most recent injection to that associated with previous injection(s) or the calibration measurement.

With continued reference to FIG. 4, a performance monitoring system 600 is in operative communication with the electronic control device 400. The performance monitoring system 600 may be configured to monitor, log, and analyze key system performance metrics to allow for predictive maintenance, spotting negative trends in manufacturing and assembly processes, and recursively update system algorithms with single site or networked data to improve performance. In some examples or aspects, the performance monitoring system 600 may be configured to perform calculations on operation data to determine information about the performance, life, or operation of one or more components of the fluid injector system 100. In further examples or aspects, the performance monitoring system 600 may be configured assess the performance, life, or operation of one or more components of the fluid injector system 100 that is otherwise not able to generate operation data. For example, the performance monitoring system 600 may be configured to compare gathered operation data with stored operation data, a hardcoded threshold, or a dynamic threshold. The comparison can be performed with operation data from one or more fluid injector systems 100 in a network of a plurality of fluid injector systems 100.

In some non-limiting embodiments or aspects, the performance monitoring system and/or the electronic control device 400 may also be in operative communication with a remote system 610, such as via a network 620. The performance monitoring system 600, the electronic control device 400, and the fluid injector system 100 may interconnect (e.g., establish a connection to communicate) via wired connections, wireless connections, or a combination of wired and wireless connections.

In some non-limiting embodiments or aspects, the performance monitoring system 600 includes one or more devices capable of receiving data and/or information (e.g., operation data, maintenance action, etc.) from one or more sensors 540, 550, 560 of the fluid injector system 100 and/or the remote system 610 via the network 620 and/or communicating data and/or information (e.g., operation data, maintenance action, etc.) to the fluid injector system 100 and/or the remote system 610 via the network 620. In some non-limiting embodiments or aspects, the performance monitoring system 600 is in communication with a data storage device, such as the memory 408 of the electronic control device 400. In some non-limiting embodiments or aspects, the performance monitoring system 600 may be implemented within fluid injector system 100 and/or the remote system 610.

In some non-limiting embodiments or aspects, the remote system 610 may include one or more devices capable of receiving data and/or information (e.g., operation data, maintenance action, etc.) from the performance monitoring system 600 and/or the fluid injector system 100 via the network 620 and/or communicating data and/or information (e.g., operation data, maintenance action, etc.) to the performance monitoring system 600 and/or the fluid injector system 100 via the network 620. For example, the remote system 610 may include a computing device, such as a server, a group of servers, and/or other like devices. In some non-limiting embodiments or aspects, the remote system 610 may be implemented by or on behalf of an original equipment manufacturer (OEM) of the fluid injector system 100 (e.g., an OEM of one or more components or devices of the fluid injector system 100, etc.), a provider of the fluid injector system 100, an imaging site or a hospital including the fluid injector system 100, a service technician assigned to the fluid injector system 100, and/or the like.

The network 620 may include one or more wired and/or wireless networks. For example, the network 620 may include a cellular network (e.g., a long-term evolution (LTE) network, a third generation (3G) network, a fourth generation (4G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, a short range wireless communication network (e.g., a Bluetooth network, a near field communication (NFC) network, etc.) and/or the like, and/or a combination of these or other types of networks.

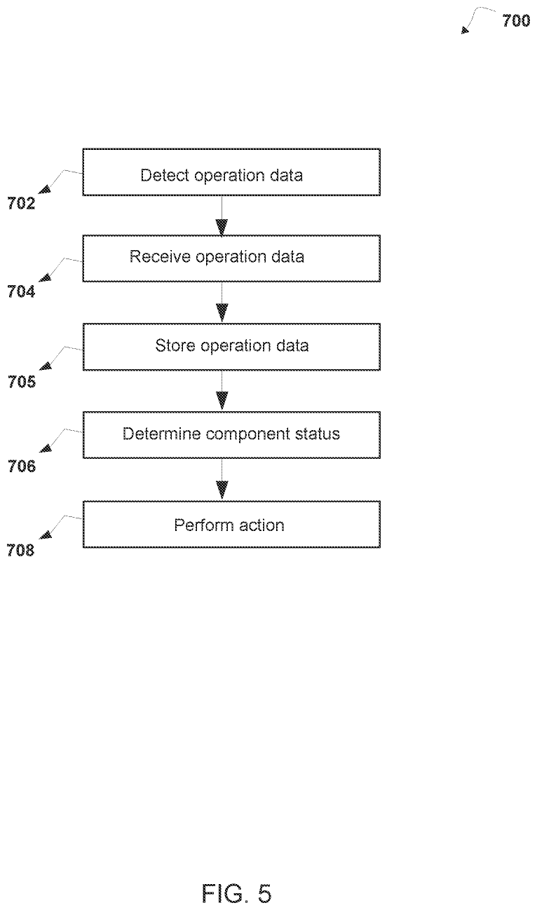

With reference to FIG. 5, a flowchart of a non-limiting embodiment or aspect of a process 700 for performance monitoring of a fluid injector system is shown. In some non-limiting embodiments or aspects, one or more of the steps of the process 700 are performed (e.g., completely, partially, etc.) by the performance monitoring system 600. In some non-limiting embodiments or aspects, one or more of the steps of the process 700 are performed (e.g., completely, partially, etc.) by another device or a group of devices separate from or including the performance monitoring system 600, such as the fluid injector system 100 (e.g., one or more devices of fluid injector system 100, etc.) and/or the remote system 610 (e.g., one or more devices of the remote system 610, etc.).

With continued reference to FIG. 5, at step 702, the process 700 includes detecting operation data during operation of the fluid injector system 100. For example, the performance monitoring system 600 may use one or more of the sensors, such as one or more of the sensors 540, 550, 560, to detect operation data during operation of the fluid injector system 100. Such operation data may be generated, for example, during a signal exchange of data between various components of the fluid injector system 100. In some non-limiting embodiments or aspects, the one or more of the sensors, such as one or more of the sensors 540, 550, 560, detect operation data in a continuous manner, in a periodic manner, in response for a user request for a performance analysis, a diagnostic operation, and/or a benchmarking service to be performed in association with the fluid injector system 100, automatically in response to a boot-up operation and/or another operation performed by the fluid injector system 100, and/or the like.

In some non-limiting embodiments or aspects, operation data includes one or more operation parameters associated with one or more operations of the fluid injector system 100. For example, operation data can include one or more operation parameters associated with at least one of: one or more drive components of the fluid injector system 100, at least one disposable component configured for use with the fluid injector system 100, and at least one administration line configured for use with the fluid injector system 100. Operation data may be data acquired during a fluid delivery procedure, a priming procedure, a calibration procedure, during a resting or dormant state of the fluid injector system 100, during user interaction with the fluid injector system 100, and any combination thereof.

In some non-limiting embodiments or aspects, an operation parameter can include at least one of the following parameters associated with the fluid injector system 100: a flow rate during one or more injections (e.g., a maximum, a minimum, an average, a total, etc. detected by one or more flow rate sensors), a volume pumped and/or delivered during one or more injections (e.g., a maximum, a minimum, an average, a total, etc.), a duration of time of one or more injections (e.g., a maximum, a minimum, an average, a total, etc.), a difference between the flow rate during the one or more injections and a programmed flow rate (e.g., set by an injection parameter of an injection protocol, etc.) of the one or more injections, a difference between the volume pumped and/or delivered during the one or more injections and a programmed volume to be pumped and/or to be delivered (e.g., set by an injection parameter of an injection protocol, etc.) during the one or more injections, a number of injections performed, an achieved pressure of one or more injections (e.g., a maximum, a minimum, an average, a total, etc.), a difference between the achieved pressure of the one or more injections and a programmed pressure to be achieved (e.g., set by an injection parameter of an injection protocol, etc.) during the one or more injections, a duration of time powered-on (e.g., a maximum, a minimum, an average, a total, etc.), a number of times power has been cycled, an energy consumption (e.g., a maximum, a minimum, an average, a total, etc.), a linear amount of power delivered or used (e.g., an integral of ((pressure)*(flow rate))/(time), etc.), a non-linear amount of power delivered or used (e.g., an integral of a f(pressure)*(time), etc.), a voltage (e.g., a maximum, a minimum, an average, a total, etc.), a resistance, a current, a noise or signal level, a mechanical force produced, and/or the like (e.g., a maximum, a minimum, an average, a total, etc.) of one or more drive components of the fluid injector system, an existence or operability of communications with one or more other systems or devices, a number and/or a type of error codes received, a number, a duration, and/or a type of user interface keys actuated (e.g., pressed, etc.), power line conditions, a temperature and/or a humidity within an fluid injector system (e.g., a maximum, a minimum, an average, a total, etc.), a temperature and/or a humidity of an environment surrounding an fluid injector system (e.g., a maximum, a minimum, an average, a total, etc.), a vibration frequency and/or amplitude (e.g., a maximum, a minimum, an average, a total, etc.), a movement above a threshold movement (e.g., as measured by an accelerometer, etc.), a number of times cleaned, a staff rating of wear (e.g., a numerical rating, etc.), a staff rating of cleanliness (e.g., a numerical rating, etc.), a service record of service (e.g., a number of services performed, a type of services performed, etc.), a system rating of wear (e.g., a numerical rating, etc.), a system rating of cleanliness (e.g., a numerical rating, etc.), a number of one or more disposables (e.g., syringes, transfer sets, etc.) sold to and/or used by an associated customer, an amount of contrast used, a type of contrast used, a vial size of contrast used, a number of fluid injector systems at an imaging site including the fluid injector system, a turnover rate of users or operators associated with an imaging site including the fluid injector system, an identifier of a user or operator associated with one or more operations or uses of the fluid injector system, an identifier of a customer associated with an imaging site including the fluid injector system, an indication of liquid within the fluid injector system (e.g., as detected or measured by one or more liquid sensors, etc.), an amount (e.g., a maximum, a minimum, an average, a total, etc.) of x-ray radiation, RF exposure, magnetic field exposure, and/or the like in an environment surrounding the fluid injector system, one or more injection protocols used for one or more injections, and/or the like.

With continued reference to FIG. 5, at step 704, the process 700 includes receiving the operation data gathered from one or more of the sensors, such as one or more of the sensors 540, 550, 560. For example, the performance monitoring system 600 receives operation data from one or more of the sensors associated with the fluid injector system 100. In some non-limiting embodiments or aspects, the performance monitoring system 600 receives operation data from the one or more sensors in a continuous manner, in a periodic manner, in response for a user request for a performance analysis, a diagnostic operation, and/or a benchmarking service to be performed in association with the fluid injector system 100, automatically in response to a boot-up operation and/or another operation performed by the fluid injector system 100, and/or the like.

With continued reference to FIG. 5, at step 705, the process 700 includes storing operation data from one or more of the sensors, such as one or more of the sensors 540, 550, 560. For example, the performance monitoring system 600 may be configured to store the collected operation data locally on the fluid injector system 100, such as in the memory 408, for analysis and determination of actions to be taken. Alternatively, or in addition, the performance monitoring system 600 may be configured to store the collected operation data remotely, such as in a remote memory, for analysis and determination of actions to be taken. Data may be stored in a database, a table, a library, or any other form of storage.

With continued reference to FIG. 5, at step 706, the process 700 includes determining a component status of at least one component of the fluid injector system 100. In some examples or aspects, determining the component status of at least one component of the fluid injector system 100 may include determining a status for at least one of: the one or more drive components (such as the one or more drive components 510a, 510b, 510n), the at least one disposable component (such as one or more fluid reservoirs 500a, 500b, 500n), and the at least one administration line (such as the at least one administration line 530). In some examples, the performance monitoring system 600 may determine a component status by comparing the received operation data with stored operation data. The performance monitoring system 600 may determine component status based on the operation data in a continuous manner, in a periodic manner, in response for a user request for a predictive maintenance analysis, a diagnostic operation, and/or a benchmarking service to be performed in association with the fluid injector system 100, automatically in response to a boot-up operation and/or another operation performed by the fluid injector system 100, and/or the like.

In some non-limiting embodiments or aspects, the component status includes one or more predictions of an operation failure or a misuse of at least one component of the fluid injector system 100. Such predictions can be used for predicting system maintenance, spotting negative trends in manufacturing and assembly processes, and recursively updating system algorithms. For example, the one or more predictions of an operation failure or a misuse may provide an indication (e.g., a score, a number, a ranking, a probability, a likelihood, etc.) of an operation failure occurring in or misuse of any component of the fluid injector system 100. As an example, an operation failure or misuse may include a failure or a misuse of the fluid injector system 100 (e.g., a failure or a misuse of one or more operations of the fluid injector system 100, a failure or misuse of one or more devices and/or one or more components of one or more devices of the fluid injector system 100, etc.). In such an example, an operation failure of the fluid injector system 100 may include at least one of the following: a software failure, a hardware failure, a component or device failure, and/or the like that causes the fluid injector system 100 to operate contrary to one or more predefined operation thresholds. For example, an operation failure may require service, repair, and/or replacement of the software, the hardware, the component or device, and/or the like affected by the operation failure in order for the fluid injector system 100 to operate in a proper manner.

With continued reference to FIG. 5, at step 708, the process 700 includes performing at least one action based on the one or more operation failures or misuses. As an example, performance monitoring system 600 may perform at least one maintenance action for a user or operator of the fluid injector system 100 (e.g., via a user interface provided by output component), to the fluid injector system 100, to the remote system 610, to a computing system implemented by or on behalf of a provider of the fluid injector system 100, to a computing system implemented by on or behalf of an imaging site, a customer, or a hospital, etc.), and/or the like.

In some non-limiting embodiments or aspects, the action may be a prompt for a user or operator to perform one or more maintenance actions, an instruction that causes the fluid injector system 100 to perform one or more maintenance actions, an indication that one or more maintenance actions have been scheduled to be performed for and/or with the fluid injector system 100, an indication that one or more maintenance actions have been performed for and/or with the fluid injector system 100, a list of other fluid injector systems of a plurality of fluid injector systems at an imaging site including the fluid injector system 100, and/or one or more maintenance agreements associated with the fluid injector system 100.

In some non-limiting embodiments or aspects, the action includes an instruction that causes the fluid injector system 100 to automatically perform one or more maintenance actions. For example, a maintenance action includes at least one of the following actions performed automatically with the fluid injector system 100 (e.g., with one or more components or devices of the fluid injector system 100, etc.): providing a prompt for a user (e.g., via a user interface of output component, etc.) to perform one or more maintenance actions for the fluid injector system 100 (such as a self-maintenance action), scheduling a service technician (e.g., dispatching a service technician to the fluid injector system 100, etc.) to repair, service, and/or replace the fluid injector system 100, automatically placing an order for one or more disposables (e.g., syringes, transfer sets, etc.) and/or one more contrast agents, providing instructions (e.g., via a user interface of output component, etc.) for a user or operator to use the fluid injector system 100 in specific manner to avoid a specific operation failure and/or misuse of the fluid injector system 100, providing a recommendation (e.g., via a user interface of output component, etc.) to improve service based on a comparison of the fluid injector system 100 to one or more other fluid injector systems, providing (e.g., via a user interface of output component, etc.) a volume used, a volume remaining, a pressure limit, and/or the like associated with the fluid injector system 100, offering (e.g., via a user interface of output component, etc.) a service plan based on usage-based operation parameters of the fluid injector system 100, offering (e.g., via a user interface of output component, etc.) a customized preventative maintenance service (e.g., cleaning, calibration of a power supply, motors, etc.), recommending (e.g., via a user interface of output component, etc.) training for a user or operator, rebooting software, updating software, transmitting operation data and/or an alert to the remote system 610, measuring component degradation, wear, or cleanliness, providing remote entry to a remote computing system to modify and/or update software and/or one or more operation parameters, disabling or limiting one or more operations or functions (disabling injections with operation parameters that define a flow rate that would exceeds a threshold flow rate and/or a pressure that would exceeds a threshold pressure, etc.), disabling power, stopping an injection, cycling power, prompting a customer to transmit a request for service directly from the fluid injector system 100, and/or the like.

In some non-limiting embodiments or aspects, the action includes performing a predefined sequence of operations and detecting any deviation in any of the steps of the sequence from a predetermined threshold. For example, prior to installing the one or more disposable components, such as one or more fluid reservoirs 500a, 500b, 500n, the performance monitoring system 600 may perform at least one action to check the operating status of the one or more drive components, such as the one or more drive components 510a, 510b, 510n. In some examples, the performance monitoring system 600 may monitor the motor current under a no-load condition (e.g., by receiving operation data from the at least one force sensor 560 when the one or more drive components 510a, 510b, 510n are not acting on the one or more fluid reservoirs 500a, 500b, 500n). The operation data may be recorded and saved in the memory of the electronic control device 400 and compared against a predetermined threshold for the no-load motor current condition. Deviations outside the predetermined threshold and/or trending outside of a selected moving average window may result in the performance monitoring system 600 providing at least one maintenance action, such as signaling an alert to the user (such as via the output component) to contact service for potential repair or replacement of a motor module. In some examples, a service call may be automatically scheduled. In further examples, the performance monitoring system 600 may cause the fluid injector system 100 to be operated in a manner indicated by the component status, such as by reducing a maximum current load, until a repair or replacement is performed. In further examples, the action may include automatically stopping or preventing operation of the fluid injector system 100 in response to the component status deviating from the predetermined threshold. Small deviations in key performance metrics can be used to spot non-catastrophic issues that can be fixed at a convenient time, such as when other equipment in the imaging suite is scheduled for service, in order to avoid costly unplanned downtime.

In some non-limiting embodiments or aspects, the action may include repeating at least a portion of operation of the fluid injector system during which components status indicates an operation failure or misuse. For example, if the performance monitoring system 600 indicates that a disposable component fails a leakage test during a priming operation, the performance monitoring system 600 may perform a maintenance action by repeating the leakage test to determine whether the same failure occurs again.

In some non-limiting embodiments or aspects, the action may include monitoring a viable life of one or more components of the fluid injector system 100, the at least one disposable component, such as the at least one disposable component 500a, 500b, 500n, and/or the at least one administration line, such as the at least one administration line 530. In some examples, the at least one disposable component may be pressurized to a predetermined pressure, which is then maintained for a predetermined period of time. By monitoring for movement of the at least one drive component of the fluid injector system 100, the performance monitoring system 600 can determine whether any leakage is occurring, such as due to wear on a seal. If leakage above a predetermined threshold is detected, the performance monitoring system 100 may alert the user to replace the at least one disposable component and/or stop/prevent operation of the fluid injector system 100 until the at least one disposable component is replaced.

In some non-limiting embodiments or aspects, the action may include monitoring a change in performance of the at least one sensor, such as the least one air sensor 550. For example, the performance monitoring system 600 may be configured to monitor an amount of light that passes through a lens of the at least one air sensor 500. If a deviation in the amount of light is detected, such as if the amount of light measured is below a predetermined threshold, the performance monitoring system 600 may prompt the user to clean the lens.

In some non-limiting embodiments or aspects, the performance monitoring system 600 may be configured to spot negative trends that may occur during repeated actions using the fluid injector system 100. For example, the performance monitoring system 600 may be configured to spot negative trends in friction of a plunger of at least one of the disposable components. During a purging operation, the plungers are advanced to remove air and prepare for filling. The friction of the plungers in each of the reservoirs can be recorded and tracked over time. By spotting increasing or decreasing trends in friction, the performance monitoring system 600 may perform a maintenance action, such as by alerting the user to replace the disposable component. The recorded trend information can be analyzed for any drift in manufacturing of the disposable components, or can trigger advance notice to support process investigations before significant field issues occur. In further examples, the performance monitoring system 600 may be configured to track use of the fluid injector system 100 (such as what buttons are pressed, the time it takes to move between screens on the user interface, order of operations, etc.) to make a determination about misuse, or inefficient use that would potentially require additional training, redesign of the system, or an adaptation of the user interface to the user to facilitate the user's interactions with the fluid injector system 100.

In some non-limiting embodiments or aspects, the performance monitoring system 600 may be configured to receive operation data from at least one sensor that is configured for detecting operating status of the fluid injector system 100. For example, such at least one sensor may be a sensor that is not used during fluid delivery operations. Instead, such at least one sensor may be provided for monitoring the performance of the fluid injector system 100 during fluid delivery operations. The at least one sensor may be configured to detect operation data during the fluid delivery operations. Such operation data may be used for determining a performance “signature” that is indicative of a desired performance of the fluid injector system 100. In some non-limiting embodiments or aspects, the at least one sensor may be an acoustic sensor configured for detecting an acoustic signature of the fluid injector system 100 during a fluid delivery procedure. In other embodiments or aspects, the at least one sensor may be a piezoelectric sensor configured for detecting a vibration signature of the fluid injector system 100 during a fluid delivery operation. In further embodiments or aspects, the at least one sensor may be a temperature sensor configured for detecting a temperature signature of the fluid injector system 100 during a fluid delivery operation. In further embodiments or aspects, the at least one sensor may be any combination of the acoustic, piezoelectric, temperature, or any other sensors. In response to determining that the “signature” of the fluid injector system 100 during a fluid delivery operation is outside of a predetermined threshold, the performance monitoring system 600 may perform a maintenance action, such as providing a maintenance instruction and/or operation to a service technician and/or a user or operator of a fluid injector system to service the fluid injector system 100.

In some non-limiting embodiments or aspects, the performance monitoring system 600 may be further configured for operating state of the electronic control device 400 and determining if the electronic control device 400 is operating below a predetermined threshold. For example, the performance monitoring system 600 may be configured to monitor performance of the central processing unit (CPU), such as by monitoring the speed of the CPU and/or a temperature of the CPU. In further examples or aspects, the performance monitoring system 600 may be configured to run a predetermined operation and measure the time it takes to perform such an operation. In response to the speed and/or the temperature being outside of a predetermined threshold, and/or in response to the time to perform a predetermined operation exceeding a predetermined limit, the performance monitoring system 600 may perform a maintenance action, such as limiting a number of CPU operations until full performance is restored. In some non-limiting embodiments or aspects, the performance monitoring system 600 may be further configured for providing a maintenance instruction and/or operation to a service technician and/or a user or operator of a fluid injector system to assist in restoring full CPU performance.

In some non-limiting embodiments or aspects, the performance monitoring system 600 may be configured to recursively update one or more algorithms used for operating the fluid injector system 100. For example, during the pressurization sequence to perform calibration for air detection, the compliance of each reservoir of the at least one disposable component and the overall system may be calculated and stored. If the compliance exceeds a predetermined threshold, significantly over time the algorithms can be recursively updated to reflect the new increased or decreased compliance of the system to ensure continued peak performance.

In some non-limiting embodiments or aspects, the performance monitoring system 600 may be further configured for peer-to-peer performance benchmarking of a network of fluid injector systems 100. For example, the performance monitoring system 600 may be configured to detect operation data from a plurality of fluid injector systems 100 within the same network. Using this operation data, the performance monitoring system 600 may be configured to perform an action, such as determine a risk (e.g., a probability, a likelihood, etc.) of failure and/or misuse of one or more fluid injector systems 100 within the network. In some non-limiting embodiments or aspects, the performance monitoring system 600 may be configured for providing a maintenance instruction and/or operation to a service technician and/or a user or operator of a fluid injector system 100 within the network if operation data indicates performance outside prescribed limits.

Although the disclosure has been described in detail for the purpose of illustration based on what are currently considered to be the most practical and preferred embodiments or aspects, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

The invention claimed is:

1. A fluid injector system configured for use in administering at least one fluid to a patient, the fluid injector system comprising:
at least one sensor configured for detecting operation data during operation of the fluid injector system, wherein the operation data includes one or more operation parameters associated with at least one of: one or more components of the fluid injector system, at least one disposable component configured for use with the fluid injector system, and at least one administration line configured for use with the fluid injector system; and
a control device comprising at least one processor programmed or configured to:
receive the operation data from the at least one sensor;
determine a component status for at least one of: the one or more components of the fluid injector system, the at least one disposable component, and the at least one administration line by comparing the received operation data with stored operation data, wherein the component status includes one or more predictions of an operation failure or a misuse of at least one of: the one or more components of the fluid injector system, the at least one disposable component, and the at least one administration line; and
perform at least one action based on the component status deviating from a predetermined threshold.

2. The fluid injector system of claim 1, wherein the at least one action includes a prompt for a user to initiate at least one maintenance action associated with at least one of: the one or more components of the fluid injector system, the at least one disposable component, and the at least one administration line.

3. The fluid injector system of claim 2, wherein the at least one maintenance action includes at least one of the following: scheduling a service of the fluid injector system, operating the one or more components of the fluid injector system in a specific manner indicated by the component status, replacing the at least one disposable component, replacing the at least one administration line, or any combination thereof.

4. The fluid injector system of claim 1, wherein the at least one action includes at least one of:
automatically scheduling at least one maintenance action in response to the component status deviating from the predetermined threshold;
automatically stopping operation of the fluid injector system in response to the component status deviating from the predetermined threshold; and
repeating at least a portion of operation of the fluid injector system during which the component status indicated an operation failure or misuse with the least one processor being further programmed or configured to store the operation data detected by the at least one sensor during the repetition of the at least a portion of operation of the fluid injector system.

5. The fluid injector system of claim 1, wherein the one or more operation parameters include a viable life rating associated with at least one of the one or more components of the fluid injector system, the at least one disposable component, and the at least one administration line, and wherein the one or more processors are further programmed or configured to:
determine the viable life rating based on at least one of the following: number of uses of the at least one disposable component, a force measurement of one or more drive components of the fluid injector system during pressurized delivery of fluid from the at least one disposable component, or any combination thereof.

6. The fluid injector system of claim 1, wherein the operation failure or misuse of at least one of the one or more components of the fluid injector system, the at least one disposable component, and the at least one administration line includes at least one of the following: failure of an electrical component, failure of a software component, failure of a mechanical component, receiving user input from a user that causes the fluid injector system to operate contrary to one or more predefined operation thresholds, or any combination thereof.

7. The fluid injector system of claim 1, wherein the at least one sensor is a temperature sensor, a vibration sensor, a humidity sensor, an acoustic sensor, an optical sensor, an ultrasonic sensor, a load/pressure sensor, a capacitive sensor, a sensor configured for detecting electromagnetic radiation, a user interface configured for accepting a user input, or any combination thereof.

8. A computer-implemented method for monitoring performance of a fluid injector system configured for use in administering at least one fluid to a patient, the method comprising:
receiving, with a control device comprising at least one processor, operation data gathered by at least one sensor during operation of the fluid injector system, wherein the operation data includes one or more operation parameters associated with at least one of: one or more components of the fluid injector system, at least one disposable component configured for use with the fluid injector system, and at least one administration line configured for use with the fluid injector system;
determining, with the control device, a component status for at least one of: the one or more components of the fluid injector system, the at least one disposable component, and the at least one administration line by comparing the received operation data with stored operation data, wherein the component status includes one or more predictions of an operation failure or a misuse of at least one of: the one or more components of the fluid injector system, the at least one disposable component, and the at least one administration line; and
providing, with the control device, at least one action based on the component status deviating from a predetermined threshold.

9. The computer-implemented method of claim 8, further comprising prompting a user to initiate at least one maintenance action associated with at least one of: the one or more components of the fluid injector system, the at least one disposable component, and the at least one administration line.

10. The computer-implemented method of claim 9, wherein the at least one maintenance action includes at least one of the following: scheduling a service of the fluid injector system, operating the one or more components of the fluid injector system in a specific manner indicated by the component status, replacing the at least one disposable component, replacing the at least one administration line, or any combination thereof.

11. The computer-implemented method of claim 8, further comprising at least one of:
automatically scheduling at least one maintenance action in response to the component status deviating from the predetermined threshold;
automatically stopping operation of the fluid injector system in response to the component status deviating from the predetermined threshold; and
repeating at least a portion of operation of the fluid injector system during which the component status indicated an operation failure or misuse and storing the operation data resulting therefrom.

12. The computer-implemented method of claim 8, wherein the one or more operation parameters include a viable life rating associated with the one or more drive components, the at least one disposable component, and the at least one administration line, and wherein the one or more processors are further programmed or configured to:
determine the viable life rating based on at least one of the following: number of uses of the at least one disposable component, a force measurement of one or more drive components of the fluid injector system during pressurized delivery of fluid from the at least one disposable component, or any combination thereof.

13. The computer-implemented method of claim 8, wherein the operation failure or misuse of at least one of the one or more components of the fluid injector system, the at least one disposable component, and the at least one administration line includes at least one of the following: failure of an electrical component, failure of a software component, failure of a mechanical component, receiving user input from a user that causes the fluid injector system to operate contrary to one or more predefined operation thresholds, or any combination thereof.

14. The computer-implemented method of claim 8, wherein the at least one sensor is a temperature sensor, a vibration sensor, a humidity sensor, an acoustic sensor, an optical sensor, an ultrasonic sensor, a load/pressure sensor, a capacitive sensor, a sensor configured for detecting electromagnetic radiation, a user interface configured for accepting a user input, or any combination thereof.

15. A computer program product for monitoring performance of a fluid injector system configured for use in administering at least one fluid to a patient, the computer program product comprising at least one non-transitory computer-readable medium comprising one or more instructions that, when executed by at least one processor, cause the at least one processor to:
receive operation data gathered by at least one sensor during operation of the fluid injector system, wherein the operation data includes one or more operation parameters associated with at least one of: one or more drive components of the fluid injector system, at least one disposable component configured for use with the fluid injector system, and at least one administration line configured for use with the fluid injector system;
determine a component status for at least one of: the one or more drive components and the at least one disposable component by comparing the received operation data with stored operation data, wherein the component status includes one or more predictions of an operation failure or a misuse of at least one of: the one or more drive components, the at least one disposable component, and the at least one administration line; and
perform at least one action based on the component status deviating from a predetermined threshold.

16. The computer program product of claim 15, wherein the at least one action includes a prompt for a user to initiate at least one maintenance action associated with at least one of: the one or more drive components and the at least one disposable component.

17. The computer program product of claim 16, wherein the at least one maintenance action includes at least one of the following: scheduling a service of the fluid injector system, operating the one or more drive components of the fluid injector system in a specific manner indicated by the component status, replacing the at least one disposable component, replacing the at least one administration line, or any combination thereof.

18. The computer program product of claim 15, wherein the at least one action includes at least one of:
automatically scheduling at least one maintenance action in response to the component status deviating from the predetermined threshold;
automatically stopping operation of the fluid injector system in response to the component status deviating from the predetermined threshold; and
repeating at least a portion of operation of the fluid injector system during which the component status indicated an operation failure or misuse with the one or more instructions, when executed by the at least one processor, further causing the at least one processor to store the operation data resulting therefrom.

19. The computer program product of claim 15, wherein the one or more operation parameters include a viable life rating associated with at least one of the one or more components of the fluid injector system, the at least one disposable component, and the at least one administration line, and wherein the one or more processors are further programmed or configured to:
determine the viable life rating based on at least one of the following: number of uses of the at least one disposable component, a force measurement of one or more drive components of the fluid injector system during pressurized delivery of fluid from the at least one disposable component, or any combination thereof.

20. The computer program product of claim 15, wherein the operation failure or misuse of at least one of the one or more components of the fluid injector system, the at least one disposable component, and the at least one administration line includes at least one of the following: failure of an electrical component, failure of a software component, failure of a mechanical component, receiving user input from a user that causes the fluid injector system to operate contrary to one or more predefined operation thresholds, or any combination thereof.
